(19) United States
(12) Patent Application Publication
(10) Pub. No.: US 2016/0375200 A1
(43) Pub. Date: Dec. 29, 2016

(54) METHODS FOR UNCAPPING AND RECAPPING A SYRINGE USING A PROTECTION DEVICE

(71) Applicant: Margherita Inventions S.r.l., SAN FELICE SUL PANARO (IT)

(72) Inventor: Carlo GIUBERCHIO, Pavia (IT)

(73) Assignee: Margherita Inventions S.r.l., SAN FELICE SUL PANARO (IT)

(21) Appl. No.: 15/262,665

(22) Filed: Sep. 12, 2016

Related U.S. Application Data
(62) Division of application No. 13/637,087, filed on Sep. 25, 2012, now abandoned.

(30) Foreign Application Priority Data
Mar. 26, 2010 (IT) MI2010A000506

(51) Int. Cl. A61M 5/32

(52) U.S. Cl. CPC A61M 5/3213; A61M 2005/3215

(57) ABSTRACT

Methods for removing a covering cap from a syringe needle and for recapping the needle, using a protection device with improved functionality, particularly for syringes, comprising a supporting body which is provided with a through hole for the insertion of the covering cap of a needle of a syringe, the supporting body being provided with at least one flat face, being arranged on the opposite side with respect to a face for the insertion of the covering cap in the hole, for the resting contact of the supporting body on a resting surface, the face lying opposite the flat face being provided with a raised perimetric containment rim, the hole having a diameter that is substantially larger than the diameter of the central portion of the covering cap and substantially smaller than the diameter of the end portion of the covering cap, a collar being provided around the hole, the collar extending from the face for the insertion of the covering cap.

### CROSS REFERENCE TO RELATED APPLICATION

This application is a divisional of U.S. patent application Ser. No. 13/637,087, filed Sep. 25, 2012, which is a National Phase Application of PCT International Application No. PCT/IB2011/051261, International Filing Date Mar. 24, 2011, claiming priority of Italian Patent Application No. MI2010A000506, filed Mar. 26, 2010, all of which are hereby incorporated by reference.

### FIELD OF THE INVENTION

The present invention relates to a protection device with increased functionality, particularly for syringes and the like.

### BACKGROUND OF THE INVENTION

In the field of medical or laboratory instruments, needle instruments are known, such as syringes and the like, which are adapted to extract and/or inject liquids by means of a needle.

Since it can happen that the liquids handled with such needle instruments contain substances that are contaminant or harmful to humans, conventional needle instruments are generally provided with a covering cap that fits over the needle of the instrument, to protect it, and which can be removably fixed to the instrument in such a way as to avoid accidental injury to the operator or to avoid the operator coming into contact with the contaminant or harmful substances when the needle is not being used for performing extraction or injection operations.

More precisely, the covering cap has a cannula shape of length substantially equal to that of the needle to be covered and with at least one end perforated so as to allow the needle of the instrument to be inserted.

In this way, the needle of the instrument, which is integral with the instrument proper, can be repeatedly inserted into and extracted from the covering cap between one extraction and the next or between one injection and the next.

Typically, the recapping operation is performed manually by the operator, holding the covering cap in one hand and the needle instrument in the other hand.

During such operation, it can happen that inadvertently, the operator misses the entry hole of the covering cap, thus sending the needle into contact with the fingers of his/her hand, leading to the contamination thereof with the liquid being handled, or simply injuring himself/herself with the point of the needle.

To overcome this drawback, protection devices are known which consist of a tubular grip which is adapted to accommodate the protective cap of the needle instruments and which is provided, at one end thereof, with a protection pan perforated at the inner cavity of the grip in order to permit the insertion into and the extraction from the covering cap between one extraction and the next or between one injection and the next.

In this way, the operator's hand that holds the covering cap, by gripping the conventional protection device by its grip, is protected by the pan so as to prevent accidental injury or contamination.

Once the needle has been extracted from the respective covering cap, the cap remains accommodated in the grip of the protection device until the next recapping.

Conventional protection devices are not devoid of drawbacks, including the fact that they cannot be placed on a resting surface, for example a worktable, in a stable position if the needle instrument is inserted with its needle in the tubular grip.

Indeed, by placing it on an edge, there is the risk that the needle instrument will come out of the grip or that it will fall off the worktable it is resting on.

### SUMMARY OF THE INVENTION

The aim of the present invention is to provide a protection device with improved functionality, particularly for syringes and the like, which enables both its stable positioning on a resting surface even with the needle applied to the needle instrument and also the recapping of the needle even with one hand only.

Within this aim, an object of the present invention consists in providing a protection device with improved functionality that is simple to implement, easy to use, and at low cost.

This aim, as well as these and other objects which will become better apparent hereinafter, are achieved by a protection device with improved functionality, particularly for syringes or the like, comprising a supporting body which is provided with a through hole for the insertion of the covering cap of a needle of a syringe or the like, characterized in that said supporting body has at least one flat face, which is arranged on the opposite side with respect to a face for the insertion of said covering cap in said hole, for the resting contact of said supporting body on a resting surface or the like, said face that lies opposite said flat face being provided with a raised perimetric containment rim, said hole having a diameter that is substantially larger than the diameter of the central portion of said covering cap and substantially smaller than the diameter of the end portion of said covering cap at the end for the insertion of said needle, a collar being provided around said hole, said collar extending from said face for the insertion of the covering cap.

### BRIEF DESCRIPTION OF THE DRAWINGS

Further characteristics and advantages of the present invention will become better apparent from the description of a preferred, but not exclusive, embodiment of a protection device with improved functionality, particularly for syringes or the like, according to the invention, illustrated by way of non-limiting example in the accompanying drawings, wherein:

FIG. 1 is a perspective view of a preferred, but not exclusive, embodiment of a protection device with improved functionality, particularly for syringes and the like, according to the invention, showing a syringe during the insertion thereof into the protection device;

FIG. 2 is a perspective view of the protection device and of the syringe shown in FIG. 1 after insertion;

FIG. 3 is a perspective view of the protection device and of the syringe shown in FIG. 1 after insertion and with the protection device placed on a resting surface in a first stable position thereof;

FIG. 4 is a perspective view of the protection device and of the syringe shown in FIG. 1 during the extraction of the needle of the syringe from the protection cap;

FIG. 5 is a perspective view of the protection device and of the cap shown in FIG. 4 after extraction and with the protection device placed on a resting surface in a second stable position thereof;

FIG. 6 is a perspective view of the protection device and of the cap of the syringe shown in FIG. 1 after extraction of the syringe from the protection cap and with the protection device placed on a resting surface in the first stable position thereof;

FIG. 7 is a perspective view of the protection device and of the syringe shown in FIG. 1 during insertion of the needle of the syringe into the protection cap by means of one hand only with the protection device placed on a resting surface in the first stable position thereof;

FIG. 8 is a perspective view of the protection device and of the syringe shown in FIG. 1 during the recapping of the needle of the syringe in the protection cap;

FIG. 9 is a perspective view of the protection device and of the syringe shown in FIG. 1 during the removal of the syringe from the protection device;

FIG. 10 is a perspective view of the protection device and of the syringe shown in FIG. 1 during the decoupling of the needle accommodated in the protection cap from the syringe;

FIG. 11 is a perspective view of the protection device and of the protection cap, accommodating the needle of the syringe, shown in FIG. 1 during the removal of the protection cap from the protection device.

### DETAILED DESCRIPTION OF THE INVENTION

With reference to the figures, the protection device with improved functionality, particularly for syringes and the like, generally designated with the reference numeral 1, comprises a supporting body 2 which is substantially disk-like.

In more detail, the supporting body 2 is also provided with a hole 3, arranged coaxially to the supporting body 2, for the insertion of the covering cap 4 of a needle 5 of a syringe 6 or the like.

As will be better described hereinafter, the hole 3 has a diameter that is substantially larger than the diameter of the central portion 4a of the covering cap 4 and substantially smaller than the diameter of the end portion 4b of the covering cap 4 at the end for the insertion of the needle 5 so as to ensure the engagement between the two.

According to the invention, the supporting body 2 has at least one flat face 7, arranged on the opposite side with respect to the face for the insertion of the covering cap 4 in the hole 3, for the resting contact of the supporting body 2 on a resting surface 8 or the like in a first stable position.

Advantageously, on the supporting body 2, on the opposite side with respect to the flat face 7, there is a face 9 provided with a raised perimetric containment rim 10.

In the center of the face 9, at the hole 3 and coaxially thereto, a collar 11 is provided, that extends from the face 9 and which acts as a guide element for vertically supporting the covering cap 4 with the supporting body 2 placed on the resting surface 8 with its flat face 7.

In more detail, the collar 11 extends substantially at right angles from the face 9 for a length that is shorter than the height of the outer perimetric rim 10, in order to permit the resting contact of the supporting body 2 on the side of the rim 10 on the resting surface 8 or the like, with the flat face 7 directed upward.

With such geometry, the collar 11 defines an abutment surface 12 upon which the flange 13 of the protection cap 4, if present, engages by contact.

If the flange 13 is not provided, the protection cap 4 having only a conicalness concordant with the direction of insertion in the hole 3, then the end portion 4b, being radially larger than the central portion 4a, engages by mechanical interference the hole 3.

The collar 11 has a length that is such as to allow the protection cap 4 to protrude for a predominant portion thereof from the face 7 of the device, when the device is lifted off the resting surface.

Operation of the protection device 1 with improved functionality, particularly for syringes and the like, is as follows.

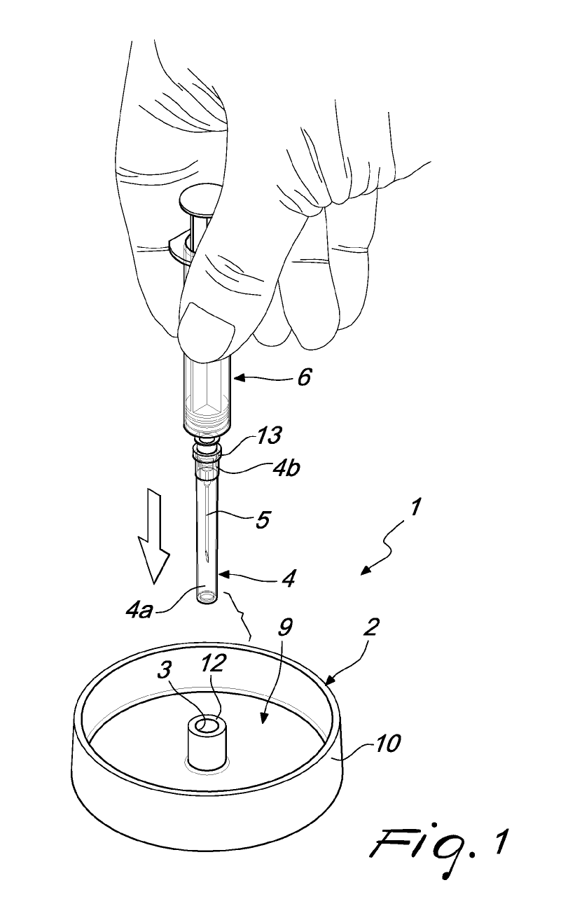

With particular reference to FIGS. 1 to 6, with the covering cap 4, which is fitted over so as to protect the needle 5 of the syringe 6, inserted in the hole 3 of the supporting body 2, it is possible to disengage the covering cap 4 from the syringe 6 by gripping it at its central portion 4a with the same hand with which the protection device 1 is supported and pulling, with the other hand, the syringe 6.

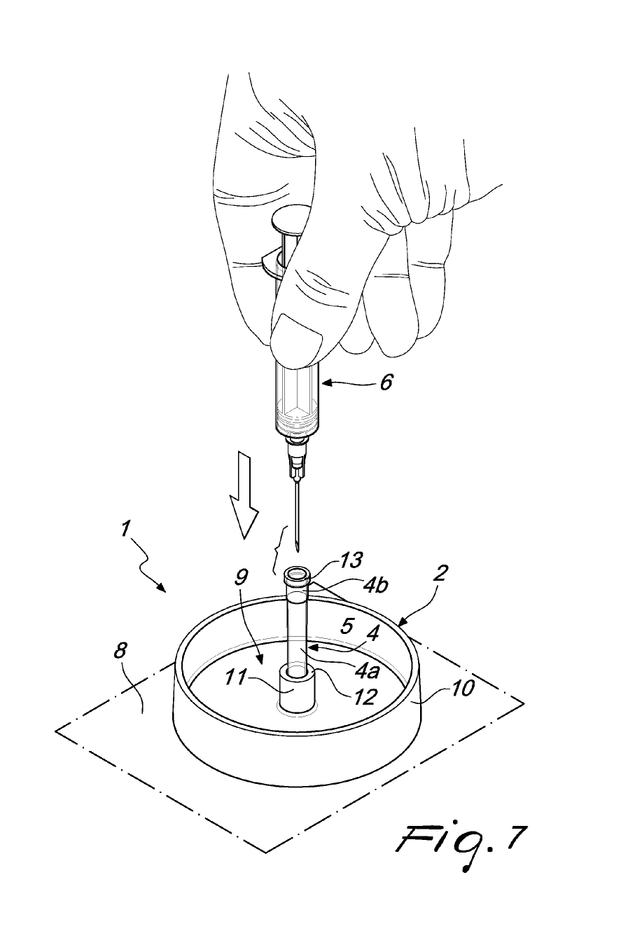

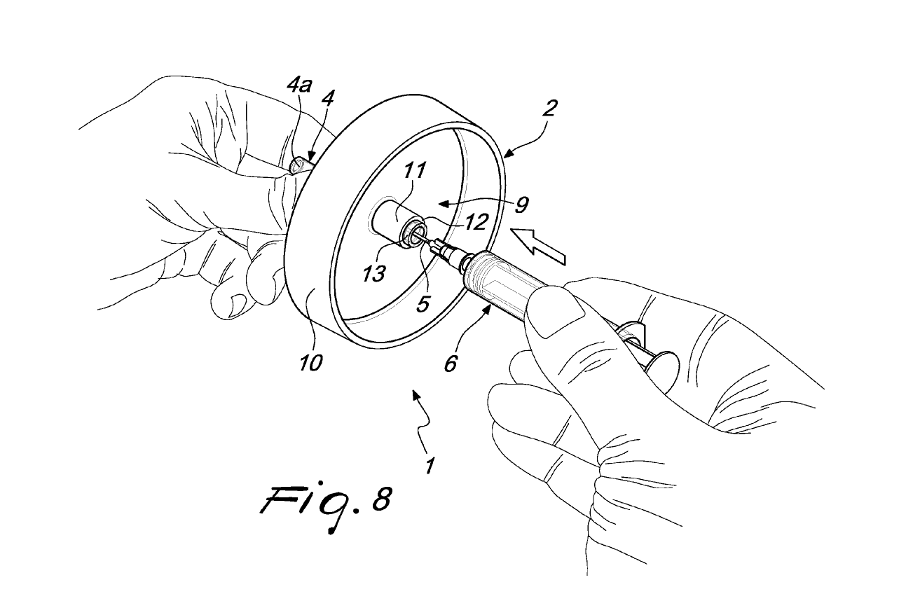

Similarly, as shown in FIGS. 7 and 8, once the withdrawal and injection operations are performed, by repeating the procedure just described in reverse it is possible to reinsert the needle 5 into the protection cap 4.

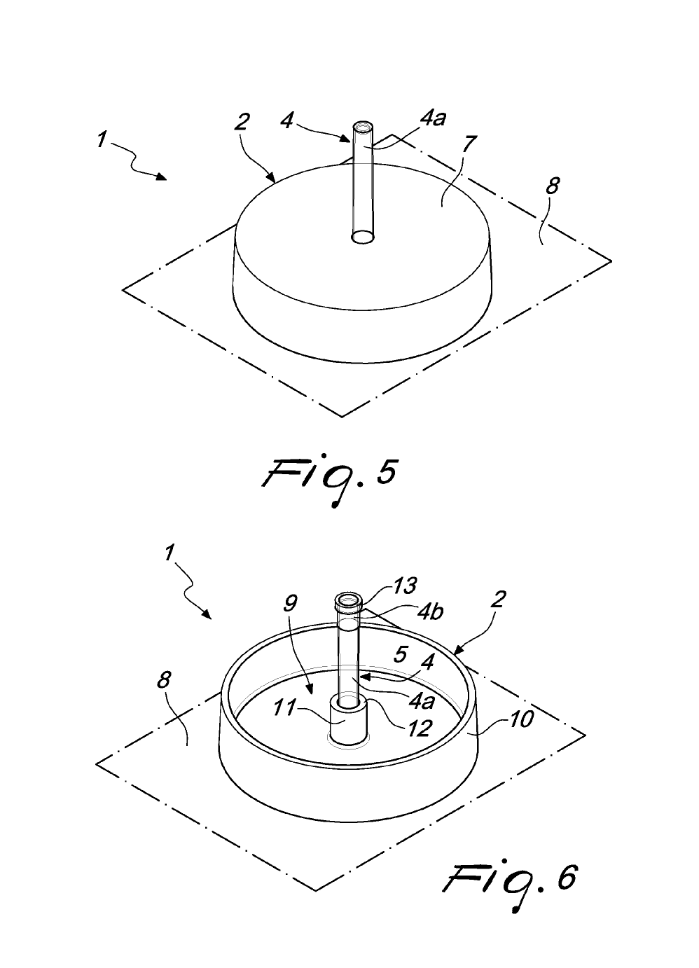

Advantageously, as shown in FIGS. 5 and 6, once the protection cap 4 is removed from the syringe 6, due to the shape of the protection device 1 it is possible to position it in two stable positions: a first in which the face 9 is turned downward, i.e. with the flat face 7 resting on the resting surface 8, and a second with the face 9 turned upward, i.e. with the outer rim 10 resting on the resting surface 8.

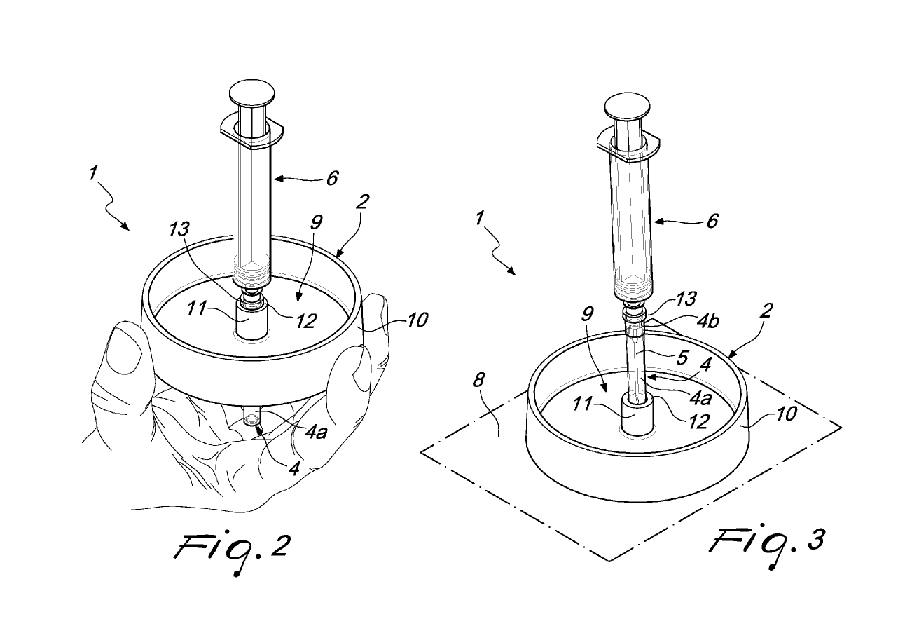

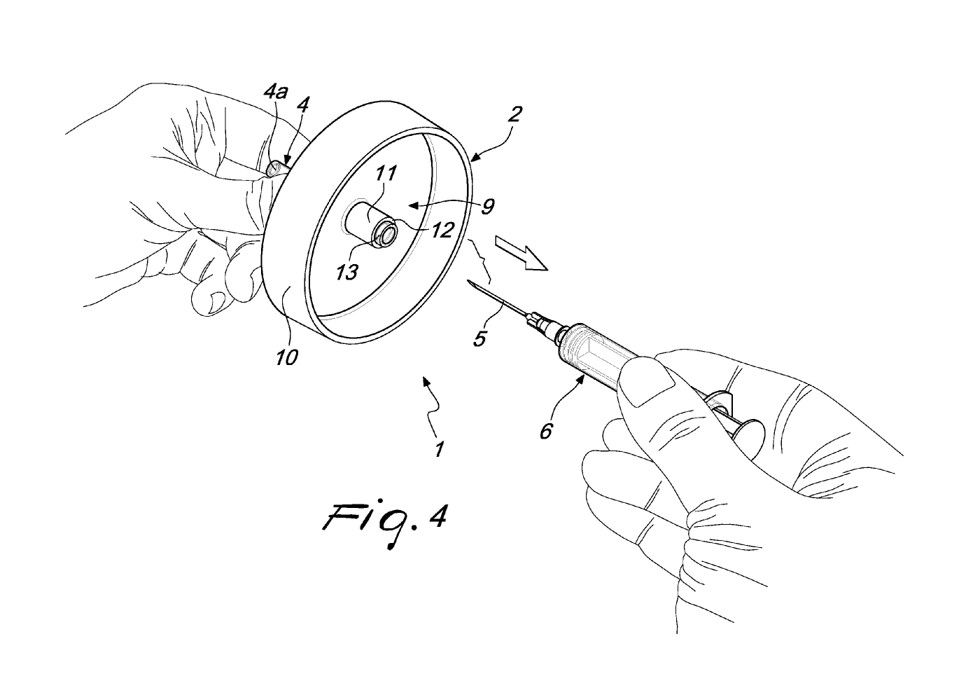

In more detail, as shown in FIG. 7, with the protection device 1 arranged in the first stable position it is possible to perform the operation of recapping the needle 5 of the syringe 6 with one hand only, thus ending up with the syringe 6 and with the needle 5 reinserted in the protection cap 4 which in turn is inserted in the hole 3; in this way, the syringe 6 is held in a vertical position thanks to the support provided by the collar 11 as shown in FIG. 3.

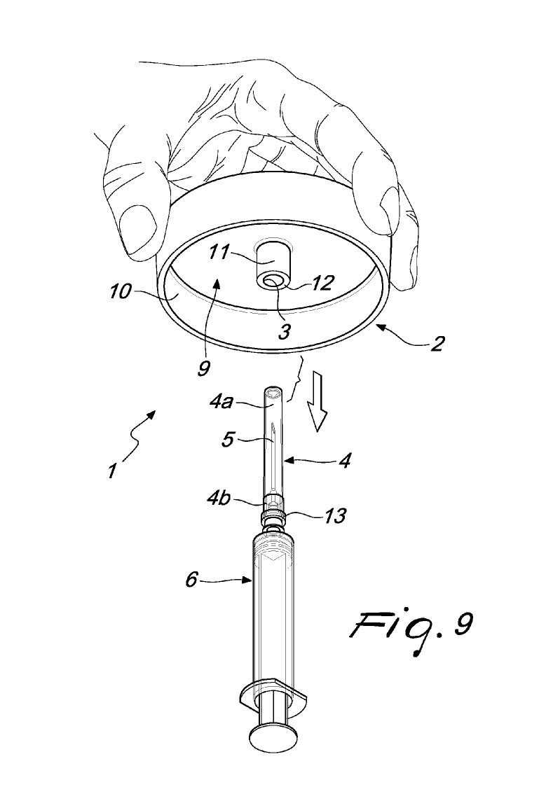

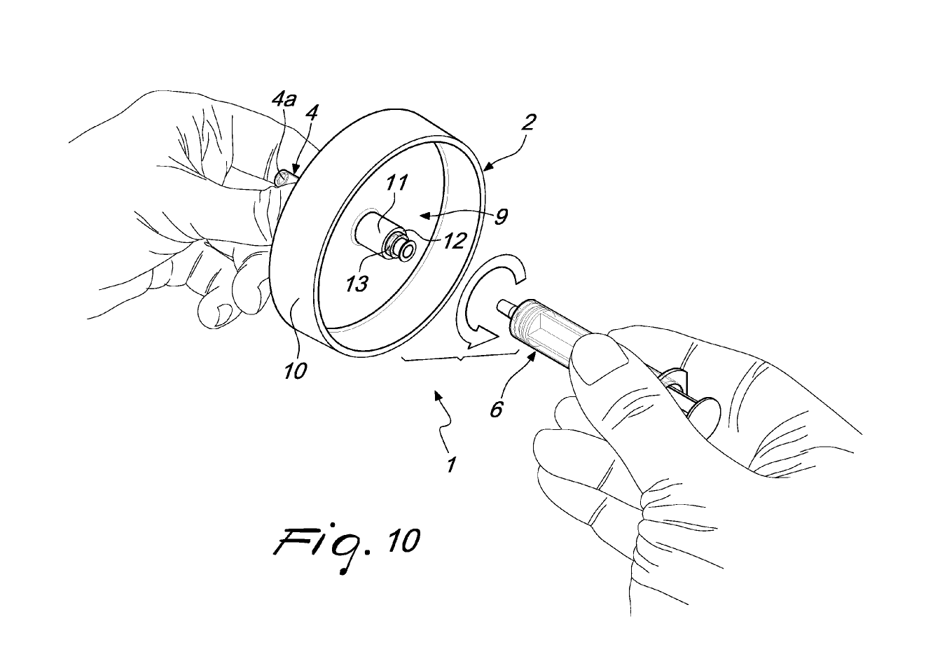

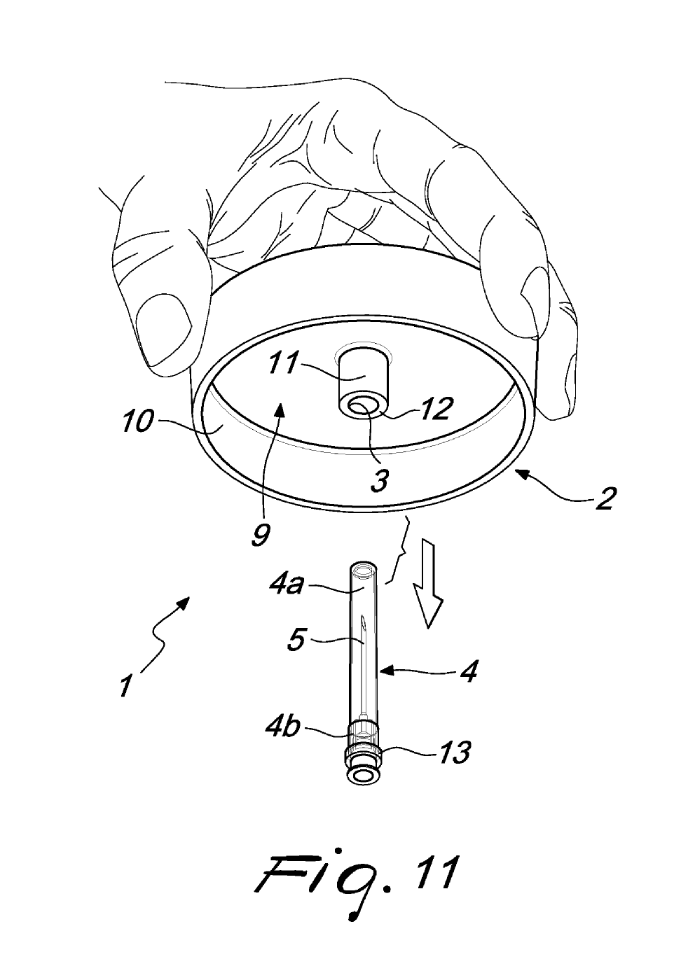

Moreover, as shown in FIGS. 9 to 11, beginning from both of the stable positions, it is always possible to decide later whether to dispose of the entire syringe 6 or whether to dispose of the recapped needle 5 separately from the rest of the syringe 6, by detaching the two parts with both hands again using the protection device 1.

In practice it has been found that the protection device with improved functionality, particularly for syringes and the like, according to the present invention, fully achieves the intended aim and object in that its geometry enables it to be arranged in two stable positions from which it is possible to perform the procedure of recapping the needle of the syringe using both hands or only one hand at the operator's discretion.

Indeed, the choice is linked to the operator's will and not to the geometric shape of the protection device, according to the invention, unlike the background art.

The choice of how to perform this operation is therefore left entirely to the operator who can change it at any time according to his/her needs.

It is not therefore a choice imposed by the device proper but one which depends only on the operator's will.

Whether using the device with one hand or with two hands the possibly contaminated surface is always the same and is never in any way in contact with either the operator or the surfaces upon which the device is rested.

By using the device according to the invention it is also possible to correctly perform a differentiated waste collection.

Such possibility relates to the use of the device as a recipient for containing other material (e.g. glass of a phial or used cotton) when it is placed on a surface for recapping with one hand only. Indeed in this case, once the needle is recapped it is possible, after lifting the device off the resting surface, to grip the cap of the syringe from below so as to prevent the contents of the device from falling and, with a small rotational movement, separate the recapped needle from the body and then dispose of the body in a first container and then, still holding the cap, it is possible, by overturning the device, to dispose of the contents in a second container and finally, by releasing the cap, to dispose of the recapped needle in a third container.

Another advantage of the protection device according to the present invention consists in that, when arranged with the rim turned upward, it is possible to use it as a recipient in which what is needed for performing the injection can be temporarily accommodated and therefore transported before performing it and, later, in which discarded material like vials, cotton or other materials can be disposed of temporarily after use, so as to not have to contaminate any other surface.

Another advantage of the protection device, according to the present invention, consists in that the flat face that comes into contact with the operator's hand or hands never comes into contact under any circumstances with parts of the syringe which are potentially contaminant, thus protecting the operator's hands from direct contact with parts of the syringe that are potentially contaminant.

Another advantage of the protection device, according to the present invention, consists in that the protruding position of the protection cap from the same side as the perimetric rim, as well as making it possible to recap the needle using two hands or only one hand, makes it more compact, and gives it a more homogeneous shape, thus making it better arrangeable and transportable in that it is free from surfaces that protrude outside the concave side of the protection device.

A further advantage of the protection device, according to the present invention, consists in that it is easier to handle and easier to clean thanks to its round shape, free from sharp corners and edges where dirt can become deposited.

Another advantage of the protection device, according to the present invention, consists in that it is economically competitive in comparison to the background art.

The protection device with improved functionality, particularly for syringes or the like, thus conceived, is susceptible of numerous modifications and variations, all of which are within the scope of the appended claims.

In addition, all the details may be replaced by other, technically equivalent elements.

In practice the materials employed, provided they are compatible with the specific use, and the contingent dimensions and shapes, may be any according to requirements and to the state of the art.

The content of the Italian patent application no. MI2010A000506, the priority of which is claimed in the present application, is incorporated as a reference.

Where the technical features mentioned in any claim are followed by reference numerals and/or signs, those reference numerals and/or signs have been included for the sole purpose of increasing the intelligibility of the claims and accordingly, such reference numerals and/or signs do not have any limiting effect on the interpretation of each element identified by way of example by such reference numerals and/or signs.

1. A method for removing a covering cap from a needle of a syringe by means of a protection device,
said protection device comprising a supporting body having a through hole configured for insertion of the covering cap, a flat face arranged on an opposite side with respect to a face for insertion of the covering cap, and a collar arranged around said hole which extends from said face for insertion that lies opposite said flat face, said face for insertion that lies opposite said flat face having a raised perimetric containment rim,
the method comprising the steps of:
grasping the protection device with a first hand about said perimetric containment rim;
holding the syringe with a second hand and inserting the needle with the associated covering cap into the hole from a side of said face for insertion, such that the covering cap protrudes from a side of the flat face; and
gripping the covering cap with the fingers of the first hand, at a side of the flat face, and removing, with the hand holding the syringe, the needle from the covering cap.

2. The method of claim 1, further comprising, after removing the needle from the covering cap, placing the protection device, with the covering cap inserted in the hole, on a resting surface, such that the perimetric rim rests on the resting surface.

3. The method of claim 1, wherein the at least one flat face never comes into contact with parts of the syringe which are potentially contaminated.

4. A method for recapping a needle of a syringe by means of a protection device,
said protection device comprising a supporting body having a through hole configured for insertion of the covering cap, a flat face arranged on an opposite side with respect to a face for insertion of the covering cap, and a collar arranged around said hole which extends from said face for insertion that lies opposite said flat face, said face for insertion that lies opposite said flat face having a raised perimetric containment rim,
the method comprising the steps of:
placing the protection device, with the covering cap inserted in the hole, on a resting surface, with the protection device turned with its flat face facing upward such that the perimetric rim rests on the resting surface and with the covering cap protruding upward from the hole on a side of said flat face;
gripping the protection device with a first hand by means of the covering cap, and inserting the needle of the syringe into the covering cap with a second hand; and
separating the syringe from the needle inserted in the covering cap.

5. The method of claim 4, further comprising disposing of the capped needle in a container for sharp waste

6. A method for recapping a needle of a syringe by means of a protection device,
said protection device comprising a supporting body having a through hole configured for insertion of a covering cap, a flat face arranged on an opposite side with respect to a face for insertion of a covering cap, and a collar arranged around said hole which extends from said face for insertion that lies opposite said flat face,
the method comprising the steps of:
placing the protection device, with the covering cap inserted in the hole, on a resting surface, with the protection device turned with its at least one flat face facing a resting surface and with the covering cap protruding upward from the collar on a side of said face for insertion;
gripping the syringe and inserting its needle into the covering cap by pushing the needle towards the face for insertion;
lifting the protection device from the resting surface so as to make the covering cap protrude from the at least one flat face whereby it is gripped by said collar;
gripping the covering cap with a first hand and, with a second hand, separating the syringe from the needle inserted in the covering cap.

7. The method of claim 6, wherein only one hand is needed to insert the needle into the covering cap.

8. The method of claim 6, wherein inserting the needle into the covering cap further comprises gripping the protection device with a different hand than the hand that grips the syringe.

9. The method of claim 6, further comprising, while still gripping the cap,
separating the recapped needle from a body of the syringe with a small rotational movement; and
disposing of the body of the syringe in a first container.

10. The method of claim 6, further comprising disposing of the capped needle in a container for sharp waste

11. The method of claim 6, wherein the at least one flat face never comes into contact with parts of the syringe which are potentially contaminated
